(19) United States
(12) Patent Application Publication
(10) Pub. No.: US 2012/0277529 A1
(43) Pub. Date: Nov. 1, 2012

(54) ENDOSCOPY CAPSULE THAT EMITS A REMOTELY VARIABLE, MAGNETIC FIELD, AND EXAMINATION APPARATUS WITH SUCH AN ENDOSCOPY CAPSULE

(76) Inventor: Stefan Popescu, Erlangen (DE)

(21) Appl. No.: 13/458,065

(22) Filed: Apr. 27, 2012

(30) Foreign Application Priority Data
Apr. 27, 2011 (DE) 10 2011 017 591.1

(51) Int. Cl. A61B 1/045

(52) U.S. Cl. 600/109

(57) ABSTRACT

An endoscopy capsule for examination and/or treatment in a hollow organ of a body has at least one magnetic element that interacts with an external magnetic field for externally controlled movement and/or rotation of the endoscopy capsule, and the magnetic field of the magnetic element can be varied by external control.

### BACKGROUND OF THE INVENTION

1. Field of the Invention

The invention concerns an endoscopy capsule for examination and/or treatment in a hollow organ of a body, of the type having at least one magnetic element that interacts with an externally applied magnetic field for externally controlled movement and/or rotation of the endoscopy capsule, as well as an examination and/or treatment device embodying at least one such endoscopy capsule and a magnetic field generation device to generate the external magnetic field.

2. Description of the Prior Art

Endoscopy capsules for examination and/or treatment of a hollow organ, in particular the gastrointestinal tract, are known that can be administered to a patient and that then move through the body by means of natural peristalsis. With such endoscopy capsules, however, there are only a few possibilities to align the field of view of an image acquisition device provided at the endoscopy capsule on a desired target, or even to suitably position instruments. Also, there must be a waiting time for natural transport through the patient to occur.

Endoscopy capsules have consequently been designed that have a permanent magnetic element that interacts with an externally applied magnetic field so as to enable an externally controlled rotation and/or translation movement of the endoscopy capsule within the hollow organ by appropriate variation of the external magnetic field.

For example, an imaging method for an endoscopy unit of the capsule type is described in U.S. Pat. No. 7,343,036. A tube is used that has field coils for generation of a static magnetic field and field gradient coils with associated gradient amplifiers to generate gradients of the external magnetic field. One field coil and one field gradient coil for each of the three Cartesian spatial coordinates are respectively provided, so that a local change of the magnetic field in all spatial directions is possible. In this way an active control to wirelessly move the endoscopy unit is achieved by the externally variable magnetic field interacting with a permanent magnet of the endoscopy unit, for example in order to guide the endoscopy unit through the gastrointestinal tract of a patient. A display device is used in order to display the images that are transmitted wirelessly by the capsule-type endoscopy unit.

A system with a magnetically guided endoscopy capsule (MGCE) has been jointly developed by Siemens AG with Olympus Medical Systems Cooperation, that allows stomach examinations to be implemented simply and comfortably, because the patient must merely swallow the endoscopy capsule. The patient then lies inside the magnetic guidance system where a magnetic field generation unit is designed to generate a variable external magnetic field. The physician uses an operating device—in particular a joystick—in order to navigate the endoscopy capsule to the regions of interest. From there the endoscopy capsule can show high-resolution images of the inside of the body in real time at a display device in the examination room.

The design of a magnetic field generation device that can be used to move a magnetic object (for example a permanent magnet of an endoscopy capsule) in an operating region is disclosed in U.S. Pat. No. 7,173,507, for example. A magnetic coil system is described therein that can generate three magnetic field components Bₓ, B_{y} and B_{z} and five magnetic field gradients. These are used in order to navigate (meaning to rotate and/or to tilt and/or to move) a magnetic object without contact. A video endoscopy capsule that is provided with a permanent magnet thus can be navigated. The magnetic endoscopy capsule tends to orient parallel to the static direction of the external magnetic field. The field gradients produce a force on the permanent magnet of the capsule, which can be described as a magnetic dipole (in this regard see also the article by David C. Meeker et al., “Optimal realization of arbitrary forces in a magnetic stereotaxis system”, IEEE Transactions on Magnetics, Vol. 32, No. 2, March 1996, Pages 320-328). Through targeted activation of the individual coils, it is possible to vary the external magnetic field and thus to orient the endoscopy capsule arbitrarily in the operating region, and moreover to exert a predefined force on it in all directions, which means that the endoscopy capsule can be rotated and moved linearly.

In WO 2009/016207 A1 a magnetic coil system is described for generation of a force on an endoscopy capsule. The magnetically directed endoscopy capsule of the system is supplemented with an imaging device (preferably a magnetic resonance device), and the magnetic field generation device is expanded so that it allows low-quality (“low end”) magnetic resonance imaging and additionally drives the endoscopy capsule with a static magnetic dipole. The magnetic field generation device should simultaneously be able to generate a stable and homogeneous magnetic field for magnetic resonance imaging, wherein the gradient coils that are used for force generation on the endoscopy capsule are also used for the magnetic resonance.

However, this known system has a number of disadvantages. The strong external basic magnetic field that is required for magnetic resonance imaging will basically flip the endoscopy capsule in the direction of the field and can thus make the endoscopy capsule navigation impossible. The positioning of the endoscopy capsule is even further complicated as soon as it is located outside of the homogeneity volume of the non-uniform basic magnetic field. An additional problem is that the strong and rapidly changing gradient fields that are used for magnetic resonance imaging alter the position of the endoscopy capsule in an unforeseeable manner. Furthermore, it applies that the permanent magnet dipole in the endoscopy capsule locally interferes with the homogeneity of the external basic magnetic field (and thus the magnetic resonance images).

The combined magnetic resonance/magnetically guided endoscopy capsule system described in WO 2009/016207 A1 consequently does not enable a navigation and magnetic resonance imaging to be allowed simultaneously. This means that the navigation of the endoscopy capsule is deactivated if magnetic resonance imaging is specifically required, and the basic magnetic field is deactivated during the navigation of the endoscopy capsule. Alternatively, it has been proposed to connect the capsule with a catheter-like tube that leads out of the hollow organ, such that the permanently magnetic dipole of the endoscopy capsule can be removed from said endoscopy capsule in order to enable magnetic resonance imaging.

Consequently, no solution has been known as of yet to reasonably integrate a magnetically navigable endoscopy capsule into magnetic resonance systems, since the basic compatibility has been in question. It would also be generally desirable to reduce the costs and the spatial requirements for a system with a magnetically guided endoscopy capsule.

### SUMMARY OF THE INVENTION

An object of the invention is to provide an endoscopy capsule and an examination and/or treatment system with such an endoscopy capsule so that the design requirements are reduced (in particular in the region of the magnetic field generation device) and magnetic resonance compatibility is provided.

To achieve this object, an endoscopy capsule of the aforementioned type according to the invention has a magnetic field of the magnetic element that can be varied with external control.

In accordance with the present invention, the navigation (thus the translation movement and/or rotation of the endoscopy capsule) is not implemented by varying an external magnetic field (in particular with regard to its direction and/or its gradient), which has a significant effort associated therewith. Instead, at least one magnetic element of the endoscopy capsule is designed so that it can itself be adjusted, such that the local magnetic field in the endoscopy capsule is varied and navigation is thereby enabled. The magnetic element is preferably realized as a coil having at least one winding, which coil can be fed with current depending on an external signal. Such a coil can develop its magnetic field depending on the current and can even be switched to be field-free without an applied current. For three orthogonal spatial directions, it is also advantageous for at least one such magnetic element (in particular thus at least one coil) to be provided to generate a magnetic field in each spatial direction.

The present invention thus enables the use of “inflexible” external magnetic fields (in particular static magnetic fields or magnetic fields having a fixed direction) in order to assist navigation of the endoscopy capsule. In particular, the external magnetic field can consequently be the basic magnetic field of a magnetic resonance device so that not only is an endoscopy capsule compatible with a magnetic resonance device achieved (which is discussed in further detail in the following), but also it is no longer necessary to provide an additional magnetic field generation device, much less one that is compatible with magnetic resonance devices, in particular if the local magnetic field of the magnetic elements can be deactivated entirely (as with a coil). A better acceptance and faster spread of such endoscopy capsules is promoted in this way. However, the costs and the spatial requirements for the examination and treatment device according to the invention are reduced overall, in particular given integration into an existing magnetic resonance device. Even if a dedicated magnetic field generation device is used, this can be designed more simply and cost-effectively because (as will be described in more detail in the following) a fixed, static magnetic field is already sufficient in order to enable a navigation of the endoscopy capsule.

The present invention is thereby based on the following considerations. The force F that is exerted on a magnetic dipole m in an external magnetic field B is

\vec{F}=grad(\vec{m}·\vec{B}).

Bₓ=B_{y}=0 applies in a homogeneous and uniform magnetic field, such that only the component B_{z} along the z-axis is not equal to 0. It follows that:

$$\vec{F} = grad ( {m}_{z} {B}_{z} ) = \frac{d ( {m}_{z} {B}_{z} )}{d x} \vec{i} + \frac{d ( {m}_{z} {B}_{z} )}{d y} \vec{j} + \frac{d ( {m}_{z} {B}_{z} )}{d z} \vec{k} ,$$

wherein the vectors i, j and k are the unit vectors respectively in the x-direction, y-direction and the z-direction. If the derivatives are solved, it follows that

$$\vec{F} = {B}_{z} ( \frac{d {m}_{z}}{d x} \vec{i} + \frac{d {m}_{z}}{d y} \vec{j} + \frac{d {m}_{z}}{d z} \vec{k} ) + {m}_{z} ( \frac{d {B}_{z}}{d x} \vec{i} + \frac{d {B}_{z}}{d y} \vec{j} + \frac{d {B}_{z}}{d z} \vec{k} )$$

or, written in a different way

\vec{F}=grad(m_{z}B_{z})=B_{z}grad(m_{z})+m_{z}grad(B_{z}).

The spatial derivatives of the dipole m and of the magnetic field B clearly correspond to gradients, such that—if the gradient associated with the dipole m is designated with g and the gradient associated with the magnetic field B is designated with G—they can also be written as

\vec{F}=B_{z}(gₓ\vec{i}+g_{y}\vec{j}+g_{z}\vec{k})+m_{z}(Gₓ\vec{i}+G_{y}\vec{j}+G_{z}\vec{k})=B_{z}\vec{g}+m_{z}\vec{G}.

However, it follows from this that it is possible in two ways to exert a force on the magnetic dipole m. First, it is possible to apply external strong gradients Gₓ, G_{y} or G_{z}, for example as are already provided within the patient receptacle of a magnetic resonance device. At the same time, a small, dipolar magnetic moment m_{z} must be activated in the endoscopy capsule so that it results as a force that:

\vec{F}=m_{z}(Gₓ\vec{i}+G_{y}\vec{j}+G_{z}\vec{k})=m_{z}\vec{G}.

However, it is also possible to activate purely local “dipole” gradients gₓ, g_{y} or g_{z} in the endoscopy capsule while the endoscopy capsule is located in a quasi-homogeneous and static magnetic field B_{z}, for example the basic magnetic field of a magnetic resonance device or a local, sufficiently homogeneous fringe field outside of the patient receptacle of a magnetic resonance device. A small local magnetic gradient within the endoscopy capsule is sufficient in order to generate a sufficient force to move said endoscopy capsule because the strong, static magnetic field B_{z} increases the force. It then results that:

\vec{F}=B_{z}(gₓ\vec{i}+g_{y}\vec{j}+g_{z}\vec{k})=B_{z}\vec{g}.

Naturally, a combination of the two cited possibilities is also conceivable, wherein all or only a few of the cited parameters are adapted in order to define the direction and the orientation of the force moving the endoscopy capsule. Here it is significant—in particular in the variant in which strong external gradients (of a magnetic resonance device, for example) are used—that is possible to deactivate the magnetic elements of the endoscopy capsule (and consequently the dipole) so that interference then no longer exists, for example in the magnetic resonance imaging.

Similar considerations can be made with regard to a rotation moment T on a magnetic dipole m in a magnetic field B, wherein

\vec{T}=\vec{m}×\vec{B}.

In a homogeneous and uniform external magnetic field with Bₓ=B_{y}=0 and the component B_{z} along the z-axis differs from zero, so that

\vec{T}=\vec{m}×B_{z}\vec{k}.

It already generally follows that, solely by adaptation of the direction of the magnetic dipole m of the endoscopy capsule, a rotation moment is generated so that the endoscopy capsule will rotate accordingly.

With regard to the force on the endoscopy capsule (ultimately the magnetic elements permanently connected with the endoscopy capsule) that directs the movement, as described above there are two approaches. For example, three orthogonal coils with adaptable currents can be used in order to determine the activation of the magnetic dipole just like its magnitude and orientation, relative to the endoscopy capsule.

A local magnetic dipole m of the endoscopy capsule then results via the superimposition of the three individual coil dipoles. This enables the navigation using the static external magnetic field B_{z} and the magnetic field gradients Gₓ, G_{y} or G_{z}, generated in particular by a magnetic resonance device inside the patient receptacle. In order to move the endoscopy capsule in such an environment, the magnetic elements (in particular the coils) are fed with current so that a local dipole m_{z} arises that is collinear with the static external magnetic field, in particular while the gradient coils of the magnetic resonance device are operated so that a gradient G arises in the desired direction; the force

\vec{F}+m_{z}(Gₓ\vec{i}+G_{y}\vec{j}+G_{z}\vec{k})=m_{z}\vec{G}

consequently arises. If the endoscopy capsule is used in a magnetic resonance device, during the magnetic resonance imaging and during other running magnetic resonance sequences the magnetic dipole m is deactivated in that all coil currents are shut off. As was already mentioned, to rotate the capsule a magnetic dipole is generated in a corresponding orientation while accounting for the direction of the external magnetic field, in particular thus the basic magnetic field of the magnetic resonance device, such that the capsule rotates in the desired direction.

However, in another embodiment at least two independently controllable magnetic elements (in particular coils) are provided to generate a local magnetic field gradient for each spatial direction. The endoscopy capsule could normally only ever be rotated if externally controllable gradient fields are not available. However, in order to nevertheless be able to produce an actuating force on the endoscopy capsule, the orthogonal coils are modified so that now at least one pair of coils (thus magnetic elements) is provided for each orthogonal axis of the local coordinate system. It is thereby possible to select the level of the current and the direction of the current separately in each coil of each pair.

For magnetic navigation of the endoscopy capsule, this is then located in a static and relatively strong external magnetic field B, wherein it is assumed again that it essentially points in a defined direction (thus that all components except for B_{z} are zero). The navigation of the endoscopy capsule is now possible even when no external gradients are present and changes in the magnitude or orientation of the static field B are very limited or not even possible. The navigation of the endoscopy capsule then takes place as follows.

The coils in each pair of coils are operated with different, in particular opposite, currents so that local gradients gₓ, g_{y} and/or g_{z} of the dipolar magnetic moment m are generated that superimpose altogether into a local gradient g that in turn interacts with the strong, static magnetic field B_{z} in order to generate a driving force along the direction of the gradient g,

\vec{F}=B_{z}(gₓ\vec{i}+g_{y}\vec{j}+g_{z}\vec{k})=B_{z}\vec{g}.

As described above, to rotate the endoscopy capsule in a defined direction a local dipole m—in particular then without gradient g—is generated in turn so that a rotation moment that rotates the capsule arises.

In passive mode, the magnetic dipole m and the local gradient g can be deactivated in that all coil currents are deactivated.

The endoscopy capsule according to the invention is consequently usable in numerous ways, for example within the patient receptacle of a magnetic resonance device using the gradients themselves that can be generated there, wherein for magnetic resonance imaging the magnetic elements can simply be deactivated. Furthermore, it is conceivable to use the endoscopy capsule according to the invention in an external fringe field of a magnetic resonance system having a particularly advantageously high field (in particular greater than 3 T); however, it is also conceivable to use a local external magnetic field that is generated by a dedicated magnetic field generation device.

The use of the fringe field of a standard magnetic resonance device (which can still have a strength of a few Tesla, even outside of the patient receptacle), which in particular applies to unshielded magnets, enables the spaces for the magnetic resonance device and the examination and/or treatment with the endoscopy capsule according to the invention to be combined, such that the patient can be driven out of the magnetic resonance device (in particular out of the patient receptacle of the magnetic resonance device), for example, in order to then be able to externally navigate the endoscopy capsule accordingly. A field map of the fringe field outside of the patient receptacle is thereby necessary that can be measured in advance within the scope of a calibration, for example, and/or can be stored in a control device of the magnetic resonance device itself.

However, as was mentioned it is also possible to use a dedicated magnetic field generation device which is provided near the patient, for example, wherein it is sufficient in the present invention to generate a static, sufficiently strong external magnetic field.

In a further embodiment of the present invention, it can be provided that the endoscopy capsule comprises an in particular radio-based communication device and/or a control unit to control the operation of the endoscopy capsule (in particular of the magnetic element) to receive external control signals for said magnetic element. In particular, the communication device is designed for bidirectional communication with an external control device, such that a data exchange is possible in both directions. For example, it is conceivable that the endoscopy capsule has an image acquisition device and/or another sensor whose data can be relayed via the communication device to an external control device, wherein radio is preferably used. Internally, the operation of the endoscopy capsule can be regulated centrally via a control unit (for example a microcontroller) that is connected accordingly with the communication device and is designed to operate the magnetic elements (in particular the coils).

In a development of the present invention, it can also be provided that the endoscopy capsule comprises at least one energy receiver for wirelessly transmitted energy to operate components of the endoscopy capsule, in particular of the magnetic elements. In this way it is thus possible to feed the energy necessary for the operation of the various systems of the endoscopy capsule (in particular the magnetic elements) wirelessly into the endoscopy capsule, for which various embodiments are conceivable. It can thereby be provided that the magnetic element itself is desired to receive energy, thus ultimately forms an energy receiver, and/or the endoscopy capsule has at least one energy storage to at least temporarily store received energy. For example, a battery and/or a capacitor can thereby be provided as an energy storage. The coils that are already provided as magnetic elements in the endoscopy capsule can likewise particularly advantageously be used in order to receive radio-frequency energy which is then stored in the at least one energy storage and can later be used in order to operate the coils, or also to feed other devices of the endoscopy capsule (an image acquisition device, for example) with current.

Furthermore, in this context the energy receiver can be a coil to receive electromagnetic energy and/or a piezoelement to receive mechanical and/or acoustic energy, wherein, as was mentioned, a coil to receive electromagnetic energy is ideally formed by the magnetic element itself.

Within the scope of the present invention, there are thereby different variants of how the energy supply of the endoscopy capsule can be realized in this wireless manner. When the endoscopy capsule is used in the patient receptacle of a magnetic resonance device, it can thus be provided that the endoscopy capsule receives the wireless, electromagnetically transmitted energy from the radio-frequency coil (for example the body coil) of the magnetic resonance device. The transmission of the radio-frequency energy can thereby take place during the magnetic resonance imaging or without imaging. In the latter case, the radio-frequency coil is temporarily used merely to transmit energy without a data acquisition taking place for the magnetic resonance imaging. Instead of the radio-frequency coil, a wireless energy transfer can be achieved electromagnetically via the gradient coil of the magnetic resonance device as well. Furthermore, given the use of a magnetic resonance device it is conceivable that the wireless energy transfer takes place via electromagnetic resonance from a dedicated energy transmission device that is also particularly advantageously used in order to supply energy to other wireless devices, for example wireless magnetic resonance acquisition coils (in particular local coils).

If the endoscopy capsule is not operated in the patient receptacle of a magnetic resonance device, it can for example be provided that the endoscopy capsule receives wireless energy via a radio-frequency transmission coil that can be placed on the body of the patient, for example. In another embodiment, the capsule can receive wireless energy via electromagnetic resonance from an energy transmission device (energy applicator) that can likewise be placed on the body of the patient.

In particular, it is conceivable and advantageous if the energy receiver in the endoscopy capsule receives the energy as mechanical and/or acoustic waves that can, for example, be generated on the surface of the body of the patient via a corresponding electromechanical actuator, for example by means of a vibrator as it is also used in magnetic resonance elastography. In this case, the energy receiver is designed as a piezoelectric transducer that transduces the acoustic and/or mechanical waves into electrical energy.

It can also be provided that the endoscopy capsule has at least one magnetic field sensor to detect the external magnetic field and/or a generated localization signal. In particular, such a magnetic field sensor can be designed as a MEMS sensor, wherein—in particular when alternating fields should be received in the form of a generated localization signal, for example, the magnetic element (in particular in the form of coils) can be used as a sensor. Received data about the external magnetic field and/or a localization signal can be used in order to determine the position and/or orientation of the endoscopy capsule. For this the sensor data are transferred (in particular via the aforementioned communication device) to an external control device which makes the necessary calculations and has also previously activated corresponding devices to transmit the localization signal.

Clearly there are thus essentially two possibilities in order to realize such a position determination of the endoscopy capsule. It can thus be provided that the endoscopy capsule measured the external magnetic field and possibly its gradient via corresponding sensors, in particular by means of an integrated MEMS magnetic field sensor. If the curve of the external magnetic field is known (such as by using a field map), where the endoscopy capsule is located can consequently be read out. For example, such a field map can be stored in the external control device.

Particular advantages result in turn when the endoscopy capsule is used within the patient receptacle of a magnetic resonance device. The endoscopy capsule can then measure electromagnetic pulses that are induced by the gradient coil (for example) in the corresponding sensors of the endoscopy capsule (preferably the coils used as a magnetic element). The measurement results—thus the sensor data—are also transmitted here to the external control device that can then determine the orientation and the position of the endoscopy capsule using the known amplitude. Such a procedure is disclosed in WO 00/13586 A1, for example, which generally refers to the position and orientation determination of objects during the magnetic resonance imaging. Alternatively, it can also be provided that radio-frequency pulses of the radio-frequency coil of the magnetic resonance device are received and measured in the endoscopy capsule.

In particular when the endoscopy capsule is used outside of the patient receptacle of a magnetic resonance device, it can also be provided that external energy transmission devices are used. For example, at least two localization signal transmitters can be used that are arranged at known positions, in particular on or above the patient body. Such localization methods are known in principle and do not need to be presented in detail here.

In addition to the endoscopy capsule, the present invention also concerns an examination and/or treatment device comprising at least one endoscopy capsule according to the invention and a magnetic field generation device to generate the external magnetic field. All embodiments with regard to the endoscopy capsule according to the invention can be analogously transferred to the examination and/or treatment device according to the invention so that the advantages already described can also be achieved with this. As was already described, it is particularly advantageous if the magnetic field generation device is a magnetic resonance device; the endoscopy capsule according to the invention can consequently be used within a common, commercially available magnetic resonance device in order to be able to link the examination and/or treatment by means of the endoscopy capsule with high-quality magnetic resonance imaging.

In a further embodiment of the examination and/or treatment device, a control device is provided that is external to the endoscopy capsule and that communicates with a control unit of the endoscopy capsule via corresponding communication devices. For example, this control device can be a central control device of a magnetic resonance device that is present in any event, which magnetic resonance device simultaneously serves as a magnetic field generation device. Such an external control device not only transmits control signals to operate the at least one magnetic element at the endoscopy capsule (in particular the control unit of the endoscopy capsule); rather it can additionally be designed to activate additional devices, for example a localization signal transmitters and/or energy transmission devices and/or the magnetic resonance device itself. In addition to this, the control device can be designed to evaluate data received from the endoscopy capsule, to the effect that the position and/or orientation of the endoscopy capsule can be determined. If the endoscopy capsule is provided with an image acquisition device or other sensor serving for the examination, their results can be suitably processed and/or visualized, in particular at a display device that can likewise be associated with the magnetic resonance device. However, it should be noted that such a control device can also be realized independently of the presence of a magnetic resonance device.

As mentioned, the control device can be designed to determine a position and/or orientation of the endoscopy capsule from sensors provided at said endoscopy capsule (in particular magnetic field sensors) and/or from sensor data received from the magnetic elements. Corresponding methods for position determination have already been discussed with regard to the endoscopy capsule, such that it can be provided (for example) that the sensor data relate to the external magnetic field and/or localization signals sent by a localization signal transmitter. For example, a field map can then be provided in the control device in order to convert measurement values with regard to the external measurement field into a position and/or orientation of the endoscopy capsule. The examination and/or treatment device can consequently also comprise at least one (in particular at least two) localization signal transmitter which emits localization signals that can be received by the endoscopy capsule, in particular by means of the magnetic elements designed as coils. However, it is particularly preferable if a radio-frequency coil of the magnetic resonance device and/or a gradient coil of the magnetic resonance device is designed to emit the localization signals and/or to modify the external magnetic field, in particular controlled via the control device. Corresponding pulses that are emitted via the radio-frequency coil and/or the gradient coil and that ultimately serve as localization signals can then be received by the endoscopy capsule, wherein the corresponding sensor data are then relayed to the control device, which control device knows the field/signal distribution resulting from the pulses, however, and can consequently determine the position and/or orientation of the endoscopy capsule (as this is described in the aforementioned WO 00/13586 A1, for example).

If a magnetic resonance device is used as a (single) magnetic field generation device, the endoscopy capsule may include an energy receiver, and the radio-frequency coil of the magnetic resonance device and/or the gradient coil of the magnetic resonance device is designed for wireless transfer of energy to the energy receiver of the endoscopy capsule, and/or a dedicated energy transmission device is provided. It is again advantageous for the magnetic elements of the endoscopy capsule (which magnetic elements are designed as coils) to themselves act as energy receivers. In this way the capsule will operate in most cases with devices that are already present if the radio-frequency coil and/or the gradient coil are used. However, this is also the case if an energy transmission device present that transmits energy in any event to an additional, wireless energy-receiving device. The magnetic resonance device can have at least one local coil with an energy receiver that is likewise designed to receive energy of the energy transmission device. The energy transmission device is consequently then provided as an energy source for multiple devices. Otherwise, the above statements with regard to the endoscopy capsule according to the invention naturally also apply.

Accordingly, the energy transmission device can be an energy transmission device that transmits the energy in the form of acoustic and/or mechanical energy. Here it is suggested that the magnetic resonance device has a vibrator for magnetic resonance elastography that can then also be used as an energy transmission device. Naturally, however, such an energy transmission device can be used independently of a magnetic resonance device, which means that it can be used with a dedicated magnetic field generation device.

In particular if the endoscopy capsule is used in a fringe field outside of the patient receptacle of a magnetic resonance device, it is advantageous for the capsule to have at least one sensor to measure the magnetic field, and for the control device to be designed to determine a position and/or orientation of the endoscopy capsule from its transmitted sensor data. However, as already described, it is generally also advantageous for the control device to be designed to operate the radio-frequency coil of the magnetic resonance device and/or the gradient coil of the magnetic resonance device and/or a localization signal transmitter, and for the determination of the position and/or orientation of the endoscopy capsule to take place dependent on this operation. With measurement of a static magnetic field (for example the fringe field of a magnetic resonance device), the control device can be designed to determine the position and/or orientation using a field map that in particular is stored in the control device.

The generation of the translation movement and the rotation of the endoscopy capsule has been described in detail with regard to the endoscopy capsule according to the invention itself. When external, strong gradients are used, the gradient coil of the magnetic resonance device is designed to generate one of the magnetic field gradients serving to move the endoscopy capsule.

### BRIEF DESCRIPTION OF THE DRAWINGS

FIG. 1 illustrates an endoscopy capsule according to the invention in a first embodiment.

FIG. 2 illustrates an endoscopy capsule according to the invention in a second embodiment.

FIG. 3 shows an examination device according to the invention in a first embodiment.

FIG. 4 shows an examination device according to the invention in a second embodiment.

FIG. 5 shows an examination device according to the invention in a third embodiment.

### DESCRIPTION OF THE PREFERRED EMBODIMENTS

In the exemplary embodiments presented in the following, use of the endoscopy capsule to examine a patient is assumed for a simpler presentation, but the exemplary embodiments can naturally be transferred accordingly to an endoscopy capsule serving to treat a patient. An examination and/or treatment device thus is encompassed within the scope of the invention.

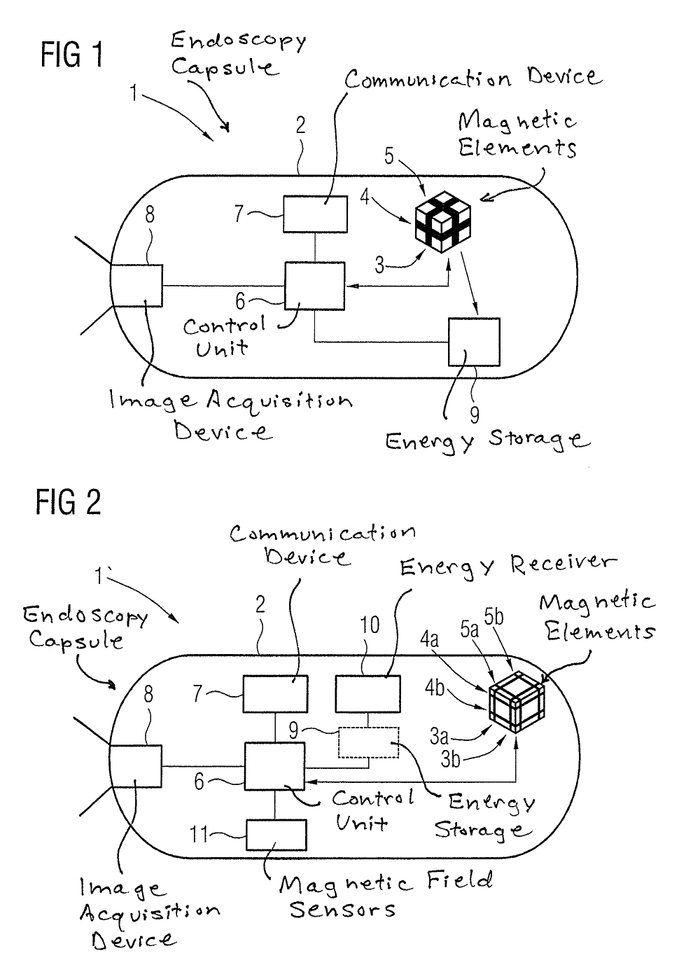

FIG. 1 shows a block diagram of a first embodiment of an endoscopy capsule 1 according to the invention. The endoscopy capsule 1 should be navigated—i.e. moved and rotated—magnetically within an external magnetic field. Therefore, the endoscopy capsule 1 has three magnetic elements within a capsule housing 2, which three magnetic elements here are designed as coils 3, 4 and 5 orthogonal to one another. The coils 3, 4, 5 can be fed with current independently of one another via a control unit 6. It is thereby possible to generate a dipole in an arbitrary direction and arbitrary strength by superimposing the fields formed by the coils 3, 4 and 5, and to deactivate said dipole by not feeding current to said coils 3, 4, 5.

The feed of current to the coils 3, 4, 5 takes place using control signals that can be received (here via radio signals) by an external control device via a communication device 7.

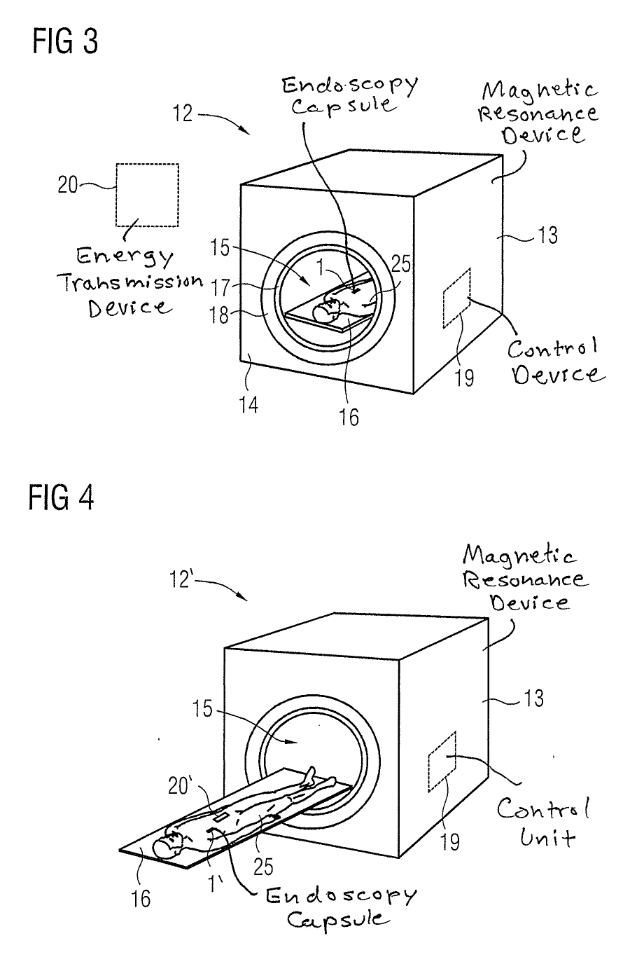

As will be explained in further detail with reference to FIG. 3, the endoscopy capsule 1 is provided for operation within a patient receptacle of a magnetic resonance device, wherein the magnetic resonance device generates via its gradient coil an external, strong gradient field that—as described above—interacts with a dipole generated via the coils 3, 4, 5 so that a force moving the endoscopy capsule 1 results. No external gradient fields are used in order to rotate the endoscopy capsule 1 into a specific orientation; rather, it is sufficient to generate a magnetic dipole moment so that it rotates in the direction of the basic magnetic field of the magnetic resonance device and thus brings the endoscopy capsule 1 into the desired attitude.

This exemplary embodiment of the endoscopy capsule 1 has an image acquisition device 8 (a camera, for example) that can then consequently be brought to corresponding locations of interest within a hollow organ of a patient (in particular in the gastrointestinal tract). The data of the image acquisition device 8 are likewise supplied to the external control device via the control unit 6 and the communication device 7, and can then be displayed at a display device.

In the endoscopy capsule according to FIG. 1, however, the magnetic elements designed as coils 3, 4 and 5 serve additional tasks. The coils 3, 4, 5 serve as energy receivers because energy in the form of electromagnetic waves can be emitted via the radio-frequency coil of the magnetic resonance device and/or the gradient coil of the magnetic resonance device, which electromagnetic waves can be received via the coils 3, 4 and 5 and be supplied to an energy storage 9. This energy storage 9 can be a battery (in particular a rechargeable battery (accumulator)) or a capacitor. Naturally, multiple energy storages can also be provided. The received energy serves for the operation of the coils 3, 4, 5 themselves, the control device 6, the communication device 7 and the image acquisition device 8. If the endoscopy capsule 1 has additional components (for example additional sensors and/or tools), these can also be operated via the received energy.

The coils 3, 4 and 5 also function as sensors for localization signals that are likewise emitted via the radio-frequency coil and/or the gradient coil, which localization signals are then likewise transmitted via the control unit 6 and the communication device 7 to the external control device 6 [sic] so that the position and orientation of the endoscopy capsule 1 can be determined because the external control device—which itself has induced the radio-frequency coil and/or the gradient coil to emit the localization signals—knows the inducement pattern and can consequently determine the position and the orientation from this and the reception data.

FIG. 2 shows an additional, modified embodiment of an endoscopy capsule 1′ according to the invention that differs from the endoscopy capsule 1 primarily in that not only one coil 3, 4 and 5 but rather two coils 3a, 3b; 4a, 4b; and 5a, 5b are provided for each of the orthogonal spatial directions here. The coils of the coil pairs 3a, 3b; 4a, 4b; 5a, 5b can be fed with current independently, in particular can also be occupied [sic] with a current flowing in opposite directions, such that gradients that in turn result in a total gradient in an arbitrary spatial direction via superimposition can be generated in each of the spatial directions. Not only arbitrary magnetic dipole moments but also local bipolar gradients can consequently be generated with the coils 3a-5b. As already described, this enables a movement of the endoscopy capsule 1′ in a static, uniform external magnetic field without the use of external gradient fields, i.e. in particular also outside of the patient receptacle of a magnetic resonance device. This is achieved via interaction of the strong, static external magnetic field with the local gradient, as has been presented above. No local gradient is necessary to rotate the endoscopy capsule 1′; here it is again sufficient to generate a magnetic dipole pointing in a specific direction.

A control unit 6 is provided in turn for controlled current feed of the coils 3a-5b; the control signals are received in turn be a communication device 7 that also serves to send data of the image acquisition device 8. However, in the present case the coils 3a-5b do not serve as energy receivers; rather, a dedicated energy receiver 10 is provided that here includes multiple piezoelements (not shown in detail for clarity). These piezoelements can receive energy transmitted in the form of mechanical acoustic waves, which energy can then be used to operate the various devices of the endoscopy capsule 1′, wherein an energy storage 9 (here drawn with dashed lines) can optionally be used in turn.

However, it is noted that other dedicated energy receivers 10 can be provided in the event the transmission of energy via electromagnetic waves, but in this case the magnetic elements can also be used.

The coils 3a-5b can furthermore be used as sensors to receive localization signals, wherein here the endoscopy capsule 1′ alternatively or additionally includes magnetic field sensors 11 executed as MEMS sensors.

The sensor data of the magnetic field sensors 11 are in turn transmitted via the communication device 7 to an external control device that can determine the position and orientation of the endoscopy capsule 1′ by means of a field map.

The various features and embodiments of the endoscopy capsules 1, 1′ can naturally be used in both endoscopy capsules, in particular those which pertain to the embodiment of the energy receiver and the sensors for position and orientation determination.

FIG. 3 shows a block diagram of a first embodiment of an examination device 12 according to the invention. The examination device 12 includes a magnetic resonance device 13 that here acts as a magnetic field generation device, wherein this is a commercially available magnetic resonance device 13. As is known, this has a basic field magnet 14 that uses superconducting coils to generate the basic magnetic field. The basic field magnet 14 has a patient receptacle 15 into which a patient bed 16 can be driven. A patient 25 who has swallowed an endoscopy capsule 1 that is now located in his or her gastrointestinal tract can be introduced into the magnetic resonance device 13 in this way.

As is receptacle 15 is surrounded by a radio-frequency coil 17 (body coil) as well as a gradient coil system 18 that, as is known, has primary coils respectively for the x-, y- and z-directions. The operation of the magnetic resonance device 13 and the complete examination device 12 are controlled via a control device 19.

A navigation of the endoscopy capsule 1 is implemented while it (in the patient 25) is located in the patient receptacle 15. For this purpose, depending on the desired movement, gradient pulses for the coils of the gradient coil system 18 and currents for the coils 3, 4, 5 of the endoscopy capsule 1 are calculated in the control device 19 so that the desired movement results via the interaction of the strong gradients of the gradient coil system 18 and the dipole that is generated by the coils 3, 4 and 5. An activation of the gradient coil system 18 is not required for a rotation; this takes place solely using the selection of a suitable local magnetic dipole moment of the endoscopy capsule 1, which then rotates in the direction of the basic magnetic field (here the z-direction), such that the dipole moment also provides for a rotation of the endoscopy capsule 1.

The control device 19 can also control the radio-frequency coil 17 and/or the gradient coil system 18 in order to transmit energy to be received by the coils 3, 4 and 5 to the endoscopy capsule 1, or to generate localization signals that are then measured by the coils 3, 4 and 5 and are again transmitted to the control device 19, which can determine the position and orientation of the endoscopy capsule 1 based on the excitation pattern (which is known to it).

In a further version of the examination device 12, it can include an optionally provided energy transmission device 20 via which energy transmission to an additional component of the magnetic resonance device 13 (for example a local coil or the like) can also take place, in addition to energy transmission to the endoscopy capsule 1 (here in the form of electromagnetic waves).

FIG. 4 shows a second embodiment of an examination device 12′ according to the invention in which the same elements are provided with the same reference characters as in FIG. 3. The magnetic resonance device 13 of the examination device 12′ that is provided—here a high-field magnetic resonance device 13—has a patient receptacle 15 and a control device 19 that also controls the operation of the entire examination device 12′, consequently the navigation of the endoscopy capsule 1′ that is used in this case. However, the navigation of the endoscopy capsule 1, which is again located in the gastrointestinal tract of a patient 25, now occurs not within the patient receptacle 15 but rather outside in the region of the fringe field of the magnetic resonance device 13. The fringe field (which here is used as the aforementioned external magnetic field) is still strong enough to enable navigation of the endoscopy capsule 1′ by, as described above, a local gradient being generated to move the endoscopy capsule 1′, which local gradient then interacts with the fringe field of the magnetic resonance device 13. In order to enable a correct movement or rotation of the endoscopy capsule 1′ at any time, a field map of the fringe field outside of the patient receptacle 15 is stored in the control device 19. This field map is also used in order to interpret the sensor data of the magnetic field sensors 11 so that a position and orientation of the endoscopy capsule 1′ can be derived from this information. Other types of position determination can naturally be used.

While the energy transmission device 20 of the examination device 12 can also be used in principle to transmit energy to the endoscopy capsule 1′, in the case of FIG. 4 an energy transmission device 20′ is used in the form of a vibrator placed on the patient 25, this vibrator also being designed for use in magnetic resonance elastography. The vibrator 20′ generates mechanical acoustic waves that are received by the piezoelements of the energy receiver 10 and transduced into electrical energy.

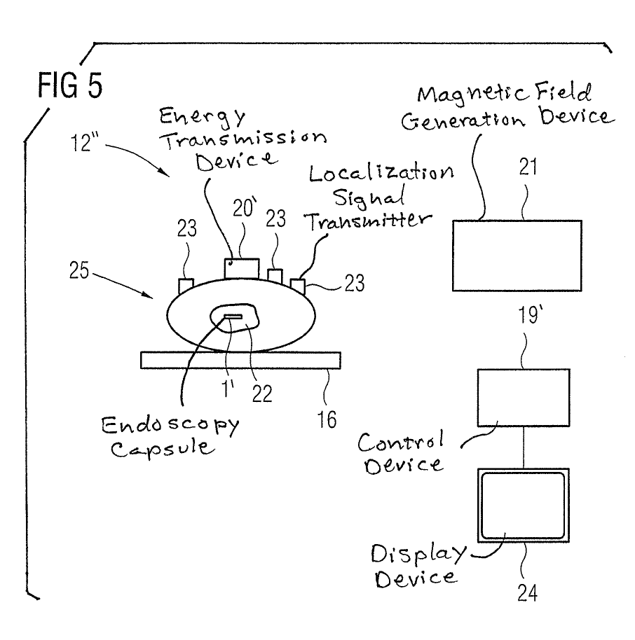

FIG. 5 shows a modified embodiment of an examination device 12″ according to the invention that has no magnetic resonance device. Nevertheless, identical components are again provided with the same reference characters for a simpler presentation. In the embodiment of FIG. 5, a dedicated magnetic field generation device 21 is used in order to generate a static, optimally uniform magnetic field for navigation of the endoscopy capsule 1′, which is again located in a hollow organ 22 (the stomach, for example) of the patient 25 positioned on a patient bed 16. Because the strength of the external magnetic field resulting via the magnetic field generation device 21 is high enough, navigation is possible as has already been described with regard to the examination device 12′. To control the operation of the examination device 12″, a control device 19′ (this time detached) is provided, which determines the corresponding control signals for current feed to the coils 3a-5b of the endoscopy capsule 1′ using a field map (again stored in the control device 19′). The energy transmission device 20′ is again provided to transmit energy to the endoscopy capsule 1′.

While the determination and orientation of the endoscopy capsule 1′ using measurement data of the magnetic field sensors 11 and with consideration of the field map would again be conceivable, here three localization signal transmitters 23 are used that are controlled by the control device 19′ so as to each emit a localization signal that can be measured (detected) by the coils 3a-5b. The corresponding sensor data can then be translated by the control device 19′ (that “knows” the precise position of the localization signal transmitters 23) into a position and orientation of the endoscopy capsule 1′.

The energy transmission device 20′ can also be designed to emit a localization signal.

A display device 24 as it is naturally also present in the other exemplary embodiments serves to display images of the image acquisition device 8.

Although modifications and changes may be suggested by those skilled in the art, it is the intention of the inventor to embody within the patent warranted hereon all changes and modifications as reasonably and properly come within the scope of his contribution to the art.

I claim as my invention:

1. An endoscopy capsule comprising:
a capsule housing configured to be swallowed by a patient and, after swallowing, to move through a hollow organ in the patient;
at least one magnetic element inside said capsule housing that generates a magnetic field that interacts with an external magnetic field to impart at least one of movement and rotation to the capsule housing within the patient; and
a control arrangement connected to said at least one magnetic element and configured to receive an extracorporeally-originating control signal and to control said at least one magnetic element to adjust said magnetic field of said at least one magnetic element and thereby control said at least one of said movement and rotation of said endoscopy housing within the patient.

2. An endoscopy capsule as claimed in claim 1 wherein said at least one magnetic element is a coil comprising at least one winding and wherein said control arrangement is configured to supply current to said coil dependent on said externally-originating signal.

3. An endoscopy capsule as claimed in claim 1 comprising three magnetic elements respectively formed by three coils, each having at least one winding, said three coils being oriented orthogonally with respect to the each other, and wherein said control arrangement is configured to supply current to each of said coils.

4. An endoscopy capsule as claimed in claim 3 wherein said control arrangement is configured to supply said respective currents to said respective coils independently of each other.

5. An endoscopy capsule as claimed in claim 1 wherein said control arrangement comprises a radio-based communication device in said capsule housing configured to receive said extracorporeally-originating control signal.

6. An endoscopy capsule as claimed in claim 1 comprising at least one energy receiver in said capsule housing, said at least one energy receiver being configured to wirelessly receive energy and to supply said energy at least to said at least one magnetic element for operation of said at least one magnetic element in generating said magnetic field.

7. An endoscopy capsule as claimed in claim 6 wherein said at least one magnetic element is directly connected to said at least one energy receiver to directly receive said energy from said at least one energy receiver.

8. An endoscopy capsule as claimed in claim 6 comprising an energy storage in said capsule housing connected between said at least one energy receiver and said at least one magnetic element, said energy storage being configured to temporarily store energy, as stored energy, received from said at least one energy receiver, and to make said stored energy available to said at least one magnetic element.

9. An endoscopy capsule as claimed in claim 6 wherein said at least one energy receiver comprises an energy receiver coil configured to wirelessly receive electromagnetic energy.

10. An endoscopy capsule as claimed in claim 6 wherein said energy receiver comprises at least one piezoelement configured to receive energy in a form selected from the group consisting of mechanical energy and acoustic energy.

11. An endoscopy capsule as claimed in claim 1 comprising at least one magnetic field sensor configured to detect at least one of said external magnetic field and a localization signal to assist in identifying a position and orientation of said capsule housing within the patient.

12. An apparatus comprising:
an endoscopy capsule comprising a capsule housing configured to be swallowed by a patient, and, after swallowing, to move through a hollow organ of the patient;
a magnetic field generator located extracorporeally of the patient that generates an external magnetic field;
at least one magnetic element inside said capsule housing that generates a magnetic field that interacts with said external magnetic field to impart at least one of movement and rotation to the capsule housing within the patient; and
a control arrangement connected to said at least one magnetic element and configured to receive an extracorporeally-originating control signal and to control said at least one magnetic element to adjust said magnetic field of said at least one magnetic element and thereby control said at least one of said movement and rotation of said endoscopy housing within the patient.

13. An apparatus as claimed in claim 12 comprising a signal source that generates said extracorporeally-originating control signals, and a communication unit in said capsule housing configured to communicate with said signal source to receive said extracorporeally-originating signals from said signal source.

14. An apparatus as claimed in claim 13 comprising at least one sensor located at said endoscopy capsule configured to inept a signal to said communication device that identifies at least one of a position or orientation of said capsule housing within the patient.

15. An apparatus as claimed in claim 14 wherein said sensor is a magnetic field sensor.

16. An apparatus as claimed in claim 13 comprising a localization signal transmitter located at said endoscopy capsule and configured to emit a localization signal to said control device.

17. An apparatus as claimed in claim 14 wherein said magnetic field generator is a magnetic resonance device.

18. An apparatus as claimed in claim 17 wherein said magnetic resonance device comprises a gradient coil system, and wherein said control device is configured to operate said gradient coil system to modify said external magnetic field.

19. An apparatus as claimed in claim 17 wherein said endoscopy capsule comprises an energy receiver at said endoscopy housing, and wherein said magnetic field generator is a magnetic resonance device comprising a gradient coil and a radio-frequency coil, and wherein said energy receiver is configured to interact with at least one of a radio-frequency field generated by said radio-frequency coil or a magnetic field generated by said gradient coil, and to convert received energy into energy that is supplied to said at least one magnetic element.

20. An apparatus as claimed in claim 19 wherein said endoscopy capsule comprises an energy storage inside said capsule housing that temporarily stores the energy received by the energy receiver, as stored energy, and supplies said stored energy to said at least one magnetic element.
